(19) United States
(12) Patent Application Publication
(10) Pub. No.: US 2014/0113385 A1
(43) Pub. Date: Apr. 24, 2014

(54) Compositions and Methods for Identifying Single Antigens or Other Molecules in Cell Preparations

(71) Applicant: Clive R. Taylor, Malibu, CA (US)

(72) Inventor: Clive R. Taylor, Malibu, CA (US)

(73) Assignee: UNIVERSITY OF SOUTHERN CALIFORNIA, Los Angeles, CA (US)

(21) Appl. No.: 13/877,436

(22) PCT Filed: Oct. 21, 2011
(86) PCT No.: PCT/US11/57393

§ 371 Date: Jan. 13, 2014

Related U.S. Application Data
(60) Provisional application No. 61/406,069, filed on Oct. 22, 2010.

(51) Int. Cl. G01N 33/543

(52) U.S. Cl. CPC G01N 33/54313
USPC 436/501; 530/391.1; 536/23.1

(57) ABSTRACT

Disclosed are compositions and methods for identifying and preferably quantifying single antigens/molecules in tissue sections and other cell preparations. The methods make use of specific antibodies or aptomers linked with beads or other micro-particles using bright field microscopy having the purpose to identify and quantify single antigens or other molecules.

### CROSS REFERENCE TO RELATED APPLICATIONS

This application claims the benefit of U.S. Provisional Application No. 61/406,069 filed Oct. 22, 2010.

### BACKGROUND OF THE INVENTION

1. Field of the Invention

The present invention provides compositions and methods for accurate detection and quantification of proteins and other molecules in tissue sections or other cell preparations. More specifically, the present invention provides compositions and methods for quantification of proteins and other molecules by labeling and counting component antigens in tissue and other cell preparations using micro-particle labeled antibodies (MiPabs).

2. Description of Related Art

The requirement for standardized and quantifiable immunohistochemistry (“IHC”) staining stems directly from the increasing use of IHC methods to identify and quantify target molecules (‘predictive’ and ‘prognostic’ or other markers) that indicate the likely success or otherwise of specific targeted therapies. Examples of such ‘predictive and prognostic markers’ include staining of receptors for HER2, estrogen (ER) and progesterone (PR) in breast cancer; but many other biomarkers currently are in development, with corresponding targeted therapies.

Currently available IHC methods are at best semi-quantitative with levels of non-reproducibility estimated at 20% or greater (College of American Pathologists 2010); completely unacceptable in terms of adverse outcomes of patients denied the proper therapy, and side effects plus costs of treatment administered inappropriately to patients unlikely to respond.

### SUMMARY OF THE INVENTION

In one embodiment, the present invention is directed to compositions and methods for identifying and preferably quantifying single antigens/molecules in tissue sections and other cell preparations. The methods make use of specific antibodies or aptomers linked with beads or other micro-particles using bright field microscopy having the purpose to identify and quantify single antigens or other molecules.

In another embodiment, the present invention provides compositions and methods for quantification of proteins and other molecules by labeling and counting component antigens in tissue and other cell preparations using micro-particle labeled antibodies (MiPabs).

Another embodiment of the present invention is directed to methods for quantifying a protein or other molecule in a tissue section or other preparation comprising the steps of (1) selecting an antibody having specificity to a protein to be identified or quantified; (2) linking the antibody to a micro-bead or other micro-particle, wherein the ratio of the number of antibodies to each micro-bead is predetermined in a defined ratio to form a micro-particle labeled antibody (MiPab) (in a preferred embodiment, the ratio is one antibody to one micro-particle); (3) applying the MiPabs to the tissue section or preparation; (4) counting the MiPabs by light microscopy with image analysis algorithms for greatest accuracy and (5) converting the MiPab count to a measured amount of protein by use of a calibration reference standard selected specifically for the protein being tested. Generally this calibration standard will be a QIRS (The methods of identifying, validating and using Quantifiable Internal Reference Standards are disclosed in PCT Application No. PCT/US2007/015417, “Quantifiable Internal Reference Standards and Uses Thereof” and U.S. patent application Ser. No. 13/174,585, filed Jun. 30, 2011, both of which are incorporated by reference in their entirety . . . ); but it may be an ‘external’ reference standard under conditions where the impact of loss of the measured protein from sample preparation is known or minimized.

In another embodiment of the present invention, a MiPab method is provided whereby antigen/molecules can be identified and quantified as individual particles (dots) at the cell membrane in formalin fixed paraffin embedded (FFPE) tissue sections. Specific examples include quantification of HER 2, EGFR and other cell surface molecules.

In another embodiment of the present invention, a MiPab method is disclosed whereby antigen/molecules can be identified and quantified based on a known fixed ratio of micro-particle-to antibody-to antigen/molecule, for example—one-to one-to one, that allows for direct counting of micro-particles to reflect direct counting of molecules, with quantification by use of a calibration standard.

### DESCRIPTION OF THE FIGURES

FIG. 1 is a schematic diagram showing single molecule 1 within the tissue section 2 carrying a single antigen site 3. The antibody 4 is attached by one of its specific binding sites 5 to the molecule 1 at antigen the antigen site 3. A single micro-particle 6 is shown chemically linked to the antibody 4. The ratio of beads to antibody to antigen molecules is 1:1:1. A count of bead number therefore translates directly to a count of the number of molecules Note that the drawing is not to molecular scale, as the bead is typically much larger relative to the specific antibody than is shown.

### DETAILED DESCRIPTION OF THE INVENTION

### Definitions

Unless otherwise indicated, all terms used herein have the meanings given below, and are generally consistent with same meaning that the terms have to those skilled in the art of the present invention. Practitioners are particularly directed to Alberts et al. (2008) Molecular Biology of the Cell (Fifth Edition (Reference Edition)) Garland Science, Taylor & Francis Group, LLC, for definitions and terms of the art. It is to be understood that this invention is not limited to the particular methodology, protocols, and reagents described, as these may vary.

As used herein, the term “antigen” is a molecule capable of stimulating a host's immune system to make a cellular antigen-specific immune response when the antigen is presented, or a humoral antibody response. An antigen may be capable of eliciting a cellular and/or humoral response by itself or when present in combination with another molecule. Exemplary antigens include but are not limited to peptides, proteins, lipoproteins, and glycoproteins.

An “antibody” is an immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′).sub.2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

As used herein, the term. “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

The term “epitope” or “antigenic determinant” or “antigen site” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

That an antibody “specifically binds” to or shows “specific binding” towards an epitope means that the antibody reacts or associates more frequently, more rapidly, with greater duration, and/or with greater affinity with the epitope than with alternative substances. As used herein, “specifically binds” means that an antibody binds to a protein with a K_{D} of at least about 0.1 mM, at least about 1 uM, at least about 0.1 uM or better, or 0.01 uM or better.

The term “specific binding site” as used herein means the portion or portion of the antibody that specifically binds to the epitope.

As used herein, the term “aptamer,” refers to oligonucleic or oligopeptide molecules that bind to a specific target molecule e.g., RNA aptamer or DNA aptamer, includes single-stranded oligonucleotides that bind specifically to a target molecule. Aptamers are selected, for example, by employing an in vitro evolution protocol called systematic evolution of ligands by exponential enrichment. Aptamers bind tightly and specifically to target molecules; preferably the aptamers to proteins bind with a K_{d} (equilibrium dissociation constant) in the range of 1 pM to 1 nM. Aptamers and methods of preparing them are described in, for example, E. N. Brody et al. (1999) Mol. Diagn. 4:381-388, the contents of which are incorporated herein by reference.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

The term “tissue” as used herein refers to is a cellular organizational level intermediate between cells and a complete organism. A tissue is an ensemble of cells, not necessarily identical, but from the same origin. Exemplary tissues include, but are not limited to, epithelial, connective, muscle, nervous, heart, lung, brain, eye, stomach, spleen, bone, pancreatic, kidney, gastrointestinal, skin, uterus, thymus, lymph node, colon, breast, prostate, ovarian, esophageal, head, neck, rectal, testis, throat, thyroid, intestinal, melanocytic, colorectal, liver, gastric, and bladder tissues. Cells may be obtained, e.g., from cell culture or breakdown of tissues. A tissue sample from a subject may include, but is not limited to, a biopsy specimen sample, a normal or benign tissue sample, a cancer or tumor tissue sample, a freshly prepared tissue sample, a frozen tissue sample, a primary cancer or tumor sample, or a metastasis sample.

One embodiment of the present invention is directed to a micro-particle labeled antibody, micro-particle labeled antibody fragments and micro-particle labeled aptamers (collectively referred to as a MiPab or MiPabs) and the use of MiPabs in the methods described herein. A MiPab generally comprises an antibody or aptamer operably linked to a microparticle, preferably a micro-bead, wherein the ratio of the number of antibodies to each micro-bead is predetermined, defined ratio. The term “operably linked” as used herein refers to positions of components so described that are in a relationship permitting them to function in their intended manner

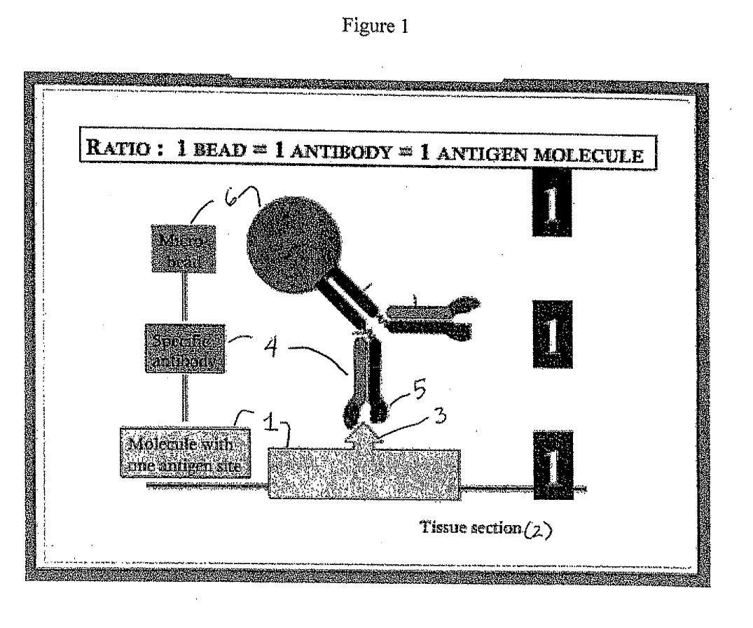

The antibody or aptamer selected should have specificity for the protein (or other molecule) to be identified or quantified. The antibodies or aptamers are preferably uni-specific with high affinity to the target protein or other molecule and limited background binding. The antigen sites (epitopes) are commonly unique conformational structures that combine with their corresponding antibody and are the targets for binding by the antibody, antibody fragment or aptamer in the MiPab complex (FIG. 1). The selected antibody (or fragment) or aptamer should bind with high affinity to its corresponding antigen site in a one to one relationship under suitable conditions. In instances where a molecule carries more than one copy of a specific antigen site it is anticipated that still only one specific antibody will bind to the molecule due to steric interference as discussed further below. Specific examples include antibodies specific for receptors for HER2, estrogen (ER) and progesterone (PR) in breast cancer. In the present invention, As used herein, the antibody “specifically binds” antibody binds to an antigen site with a K_{D} of at least about 0.1 mM, at least about 1 μM, at least about 0.1 μM or better, or 0.01 uM or better.

Micro-particles (such as beads) suitable for the present invention are commercially available. The selected beads should generally have a consistent chemical composition for linking with antibodies, and have defined controlled sizes ranging from about 0.1 to 1.0 microns. In general beads of about 0.5 microns are preferred, being visible at the lower limit of resolution of some light microscopy techniques. Beads, such as those widely used in immunologic methods, such as cell sorting by use of labeled magnetic beads, and as surface sites for localizing antibodies in fluid based immunoassays, particularly in microfluidics may be particularly useful when modified and composed for this purpose.

In a preferred embodiment each antibody molecule is linked (i.e. attached) to a single micro-particle by chemical or immunologic means. The methods for linking antibody to micro-particle (bead) in a known ratio, for example—one to one, are known to those of ordinary skill in the art. Linking of particles (e.g. beads) to the antibody may be accomplished by standard protein chemistry using commercially available beads generally suited to the purpose of protein attachment. These are generally known as microbeads with activated surfaces or already carrying bound antibodies (or other proteins). Alternatively linkage of beads to the antibody may be accomplished by immunologic binding whereby the bead is coated with immunoglobulin of the same species as the primary antibody and then attached to the bound primary antibody by a ‘linker’ antibody, a method known to those of ordinary skill in the art. See Taylor et al in 1977, for qualitative studies.

In the MiPab composition (and the methods described herein), the ratio of the number of antibodies to each micro-bead is predetermined, defined ratio. In a preferred embodiment, the ratio is one antibody to one micro-particle. Other ratios of particle to antibody to antigen molecule may be employed for quantification provided that the ratio is known and fixed for the assay. One example would be—one bead coated with multiple antibody molecules binding with a single antigen molecule; the multiple antibodies providing for increased speed of reaction and increased sensitivity. Still only one MiPab bead-antibody complex should be able to bind at the antigen site as a result of steric interference by the bead particles which are relatively very large compared to the antigen/molecule.

Another embodiment of the present invention is directed a method which comprises the steps of (1) providing an antibody having specificity for a protein (or other molecule) to be identified or quantified linked to a micro-bead or other micro-particle, wherein the ratio of the number of antibodies to each micro-bead is predetermined in a defined ratio in the form of a micro-particle labeled antibody (MiPab) (in a preferred embodiment, the ratio is one antibody to one micro-particle); (2) applying MiPabs to the tissue section or preparation; (3) counting the MiPabs in which the antibodies are linked to the protein (or other molecule), for instance by light microscopy with image analysis algorithms for greatest accuracy and (4) preferably converting the MiPab count to a measured amount of protein by use of a calibration reference standard selected specifically for the protein being tested. Generally this calibration standard will be a QIRS; but it may be an ‘external’ reference standard under conditions where the impact of loss from sample preparation in known or minimized.

The methods of the present invention include applying MiPabs to a tissue section or preparation containing the target antigen. Preferred tissue sections are formalin fixed paraffin embedded (FFPE) tissue sections. The provision of the FFPE sample is usually preceded by preparation of the FFPE cell or tissue sample. The FFPE cell or tissues sample may be prepared according to the FFPE fixation and embedding techniques commonly known to those of ordinary skill. Typically, sections of paraffin-embedded cells or tissues are obtained by (1) preserving tissue in fixative, (2) dehydrating the fixed tissue, (3) infiltrating the tissue with fixative, (4) orienting the tissue such that the cut surface accurately represents the tissue, (5) embedding the tissue in paraffin (making a paraffin block), (6) cutting tissue paraffin block with microtome in sections of 4-5 μm, and (7) mounting sections onto slides.

In an exemplary procedure, specimens used in connection with the present invention may be obtained, for instance, by fine-needle aspiration, or from the operating room by biopsy, or by more extensive therapeutic surgical procedures. Following removal of the tissue from the body, autolysis may generally be arrested by immersion in a fixative. Preferably, the fixative is formalin (in common practice a 4% solution of formaldehyde). Other fixatives may be employed. However, in a preferred embodiment, Formalin is used because it is well known to those of ordinary skill, has a long tradition of use and generally yields sufficient morphologic detail. Formalin also is inexpensive, easily stored, and universally available.

Preferably, excised tissue samples are placed directly in formalin for subsequent transportation to a suitable laboratory. Once at the suitable laboratory, for instance a surgical pathology suite (“grossing” room), the sample may be further cut, meaning that if not already sufficiently small, it is cut into small blocks to facilitate rapid penetration by the fixative (formalin penetrates relatively slowly), and placed in fresh fixative for further processing. In a preferred embodiment, time for fixation of a 5-mm-thick tissue block is about 12-24 hours, total time in fixative may vary, due to differing transportation times to the laboratory and accumulation of specimens for batch processing. Fixation time, for instance, may vary anywhere from 6-24 hours, or more.

In addition, the formalin which serves as the basis of the fixation process, may affect cell or tissue samples depending upon whether the formalin was freshly prepared and adequately buffered. There is also some variability in the rate of penetration of formalin in different types of tissues and into differently sized blocks.

Following fixation, the sample preparation preferably includes one or more process selected from embedding the tissue or cell in paraffin, de-paraffinization of the cut sections, also exposing the tissues (and therefore the analytes) to a series of chemicals and to heat. The end-result of the process of fixation, embedding and de-parrainization is a formalin-fixed paraffin embedded (FFPE) tissue section.

The FFPE samples used in connection with the present invention are subject to the same, or nearly the same, sample preparation methods. In an especially preferred embodiment, test FFPE samples containing the test analyte to be analyzed are prepared using the sample preparation methods used to generate the calibration curves associated with the Quantifiable Internal Reference Standard as described herein.

Under usual conditions the MiPab method will be employed using sufficient primary antibody to saturate antigenic sites (to ensure labeling of every molecule), with excess reagent washed clear. However where antigen is present at high density the MiPab primary antibody may be highly diluted to avoid steric interference, using statistical algorithms to calculate the total amount of antigen protein present against a calibration standard (a QIRS—quantifiable internal reference standard—as described herein).

Steric interference will occur when two MiPab conjugates attempt to bind to the same molecule bearing two or more antigen sites. Because the particles are relatively much larger than the molecule, only one can bind, other sites on the same molecule are blocked. This effect is used to maintain a one-to one-to one binding ratio of particles to molecules.

It is also possible that steric interference may occur for MiPabs seeking to bind to antigen sites on two adjacent molecules. This effect may minimized by use of the smallest visible particles, and by the use of algorithms designed to identify antigen expression above the critical threshold for the predictive test in question. For example with ER this is not a problem because sufficient anti-ER MiPabs (particles) are bound to the nucleus of a positive cell to identify it as positive even if every ER site is not occupied. Likewise for anti-HER2 MiPabs sufficient particles are bound to a cell for a particle counting algorithm to identify the cell as showing clinically significant positive ‘staining’ with a much clearer endpoint than current IHC HER 2 stains that employ different combinations of intensity, and complete membrane staining, in a scoring system that is very difficult to reproduce. Alternatively nano-beads (30-50 nm diameter may be employed in a known ratio to antibody allowing visualization of dense antigen protein deposits by aggregation of sufficient numbers of beads to become visible.

The methods of the present invention include counting the MiPabs in which the antibodies are linked to the protein (or other molecule). When a section containing the test antigen (protein to be identified or quantified) is treated with the specific micro-particle linked antibody (MiPab) then substantially every micro-particle identified under light microscopy represents a single MiPab conjugate, each of which contains a single antibody, each of which is bound to a single molecule of antigen protein. Therefore counting the number of particles provides an accurate count of the number of molecules present that carry the antigen being tested; being a-one-to one-to one relationship as shown in FIG. 1. The ratio of beads to antibody to antigen molecules should generally be a fixed, known ratio. In an especially preferred embodiment, the ratio of beads to antibody to antigen molecules is 1:1:1. Micro-bead of micro-particle labeled detection probes are measured quantitatively by image analysis using detection and scoring algorithms. Preferably, the method for counting MiPabs selected is a reproducible automated digital counting in tissues known to those of ordinary skill. Manual reading may be possible for some applications, at the cost of some level of accuracy and reproducibility. Generally, any known method for reproducible automated digital counting in tissues may be used in connection with the present invention.

One advantageous aspect of this MiPab method is the use of a known ratio, typically—one micro-particle-to one antibody-to one antigen/molecule, that allows for direct counting and quantification of a test protein by counting individual particles for molecules. This capability distinguishes the MiPab method in two important ways from other known IHC quantification methods for light microscopy.
a. prior IHC methods rely upon the use of an enzyme for development of color by a chromogen, and attempt to measure intensity; there are many uncontrolled variables such as type of chromogen, concentration of chromogen, time of reaction, and temperature all of which affect intensity independent of antigen amount, providing inaccurate results.
b. measurement of intensity is a continuous variable that is impossible to quantify by naked eye (except as crude +. ++, +++ scores) and still is difficult to quantify exactly by image analysis because of section thickness, poor localization and other factors.

In contrast MiPabs (micro-particle labeled antibodies) can be counted numerically with great accuracy and reproducibility and the amount of test antigen protein directly correlates to the number of particles (beads).

In other embodiments the MiPab methods of the present invention provide quantification in fresh or specially prepared tissues or cells, where protein antigen has not been subjected to unknown loss during processing; this capability may be a special circumstance in controlled defined specimen handling/test protocols that could be designed for future targeted therapies.

It is known that protein antigens generally suffer some loss of ability for detection during sample preparation, and the exact amount of loss is generally unknown. While the MiPab method of the present invention detects antigen in a direct quantifiable manner, it only quantifies that proportion of antigen still present and detectable after all stages of tissue preparation and storage. In a one embodiment of the present invention, the MiPab method measures the antigen (protein) present in the original fresh tissue by compensating for the amount lost to preparation and storage by use of a universal reference calibration standard for each antigen (stain), as described in Quantifiable Internal Reference Standards and Uses Thereof’. PCT/US2007/015417, which is incorporated herein by reference. (Taylor 2006a and B, Taylor and Levenson 2006).

Preferably, the methods used in connection with the present invention include defined and reproducible tissue or cell preparation procedures. In order to achieve uniform sample preparation, tissue specimen used in connection with the present invention should generally be subjected to substantially identical steps of sample preparation with substantially identical chemical reagents. However, since this would require that every hospital laboratory have access to and adopt and identical procedures, this may not currently be practical in terms of logistics or expense. Alternatively, sample preparation in connection with the present invention can be ‘qualified, so as to allow some variability, but is preferably subject to a quality control (QC) protocol that allows for determination of amount of protein present at the end of preparation, with reference to the amount originally present in the fresh tissue. In effect this QC would demonstrate that sufficient of the protein is present so as to ‘qualify’ the specimen as being suitable for the assay of choice. The level of QC described can be achieved by use of Quantifiable Internal Reference Standard (QIRS). The methods of identifying, validating and using Quantifiable Internal Reference Standards PCT Application No. PCT/US2007/015417 Quantifiable Internal Reference Standards and Uses Thereof’ and U.S. patent application Ser. No. 13/174,585, both of which are incorporated by reference in their entirety. See also, Taylor 2006a and B. Taylor and Levenson 2006.

Preferably, standardized staining protocols and AR methods are utilized that are quantifiable in the sense of measurement by weight or by molecular number as opposed to inaccurate semi-quantitative assays currently in use. The use of such methods with MiPabs allows validation of effectiveness for each antigen and antibody of interest. The methods described herein lend themselves to quantification using specific antibodies or aptomers linked with beads or other micro-particles. The method employs monoclonal antibodies, with specificity confined to the target antigen/molecule, with each molecule of antibody linked directly or indirectly as described to a single signal micro-bead or other micro-particle; such that the observation by microscopy of one single bead, translates to the presence of one molecule of antibody, which in turn translates to one antigen site or molecule. This method, described under the encompassing term of ‘micro-particle labeled antibody’ (MiP-ab) method is described in detail herein.

In quantitative assays on FFPE tissues the MiPab method would generally be employed in double ‘staining’ modality with a QIRS method to provide—(1) Quality Control (QC) of the degree to which the FFPE tissue retains antigenicity and is suitable for performance of the assay and (2) to serve as the calibration standard for quantification and calculation of the amount of test protein in the original specimen (prior to protein ‘loss’ during sample preparation (Taylor 2006a and b. Taylor Levenson 2006; The methods of identifying, validating and using Quantifiable Internal Reference Standards PCT Application No. PCT/US2007/015417 Quantifiable Internal Reference Standards and Uses Thereof’ and U.S. patent application Ser. No. 13/174,585, both of which, as indicated above, are incorporated by reference in their entirety.)

The MiPab methods may be used in connection with other methods. These include:

Methods whereby antigen/molecules can be identified and quantified in cell smear and cytospin preparations;

methods whereby antigen/molecules can be identified and quantified in fresh frozen, acetone or alcohol fixed sections;

methods whereby antigen/molecules can be identified and quantified within the cell cytoplasm, or the cell nucleus, or within the extracellular matrix, as well as the cell membrane;

methods whereby more than one type an antigen/molecule can be identified and quantified simultaneously in any of the above applications by use of separate specific antibodies each attached to micro-particles of differing size, shape or color, separately counted by image analysis algorithms;

methods whereby an antigen/molecule can be identified and quantified whereby attachment of the primary antibody to the micro-particle (bead)) may be by chemical means, or by immunological methods using secondary antibody or other labeled moieties including streptavidin and derivatives, or other linker systems; and

methods whereby the antibody probe for detection of antigen protein is replaced by a micro-particle labeled nucleotide probe for detection of DNA or RNA defined sequences by In Situ Hybridization (ISH) in bright field. This method parallels current methods using fluorescent labels in dark field (FISH) or chromogenic, or silver or gold labels in bright field (CISH, SISH GoldFISH), but is novel and distinct in the use of particles giving defined ratios of particle to probe-to DNA (or RNA) target, as in—one-to one-to one. Quantification is therefore possible without complications of label development in bright field.

The compositions and methods of the present invention make it possible to achieve objectivity and quantification in IHC or in-situ hybridization (“ISH”) staining of tissue sections. In order to achieve quantification of antigens to be possible at a molecular level in tissue sections or other cell preparations methods of the present invention preferably meet the following conditions as described herein (Taylor 2006a and b, Taylor and Levenson, 2006):
A. Defined and reproducible tissue or cell preparation.
B. Strictly controlled highly sensitive methods for quantifiable demonstration of antigen/protein
C. Availability of universal reference or calibration standard for measurement of amount of protein by weight.
D. Precise and accurate reading of signals by digital image analysis

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Those skilled in the art will recognize, or be able to ascertain using no more than routine experimentation, many equivalents to the specific embodiments of the method and compositions described herein. Such equivalents are intended to be encompassed by the following claims.

### REFERENCES

The following references are either cited or may be useful for the herein. The entire disclosure of each reference is relied upon and incorporated by reference herein

Taylor C R. Quantifiable Internal Reference Standards for Immunohistochemistry; the measurement of quantity by weight. Applied Immunohistochem Mol Morph, 14, 253-259, 2006.a.

Taylor C R. Standardization in Immunohistochemistry: the Role of Antigen Retrieval in Molecular Morphology. Biotechnic & Histochemistry 81(1):3-12,2006 b.

Taylor C R, Levenson R M. Quantification of Immunohistochemistry—issues concerning methods, utility and semiquantitative assessment. Histopathology, 49:411-424, 2006.

Taylor C R, Gordon I L, Rembaum A, Russell R, Parker J W, O'Brien R L, Lukes R J. Human B lymphocytes in Giemsa stained preparations. J Immunol Methods 17:81-89, 1977.

Gordon I L, Lukes R J, O'Brien R L, Parker J W, Rembaum A, Russell R, Taylor C R. Visualization of surface immunoglobulin of human B lymphocytes using microsphere-immunoglobulin conjugate in Giemsa-stained preparations. Clin Immunol Immunopathol 8:51-63, 1977.

Taylor C R. Immunological Studies in the Diagnosis of Lymphoma. Doctor of Medicine (MD) Dissertation, Cambridge, 1980.

1. A MiPab comprising at least one antigen binding species selected from the group consisting of an antibody, an antibody fragment and an aptamer, wherein one or more of said antigen binding species is operably linked to a micro-particle in a predetermined ratio, and wherein said antigen binding species specifically binds to an antigen site of a target antigen.

2. The MiPab of claim 1, wherein the antigen binding species is an antibody.

3. The MiPab of claim 1, wherein the antigen binding species is an aptamer.

4. the MiPab of claim 1, wherein the ratio is one antigen binding species to one micro-particle.

5. The MiPab of claim 1, where in the micro-particle is a micro-bead having a diameter from about 0.1 to 1.0 microns and the micro-bead comprises surface sites for attaching the antigen binding species.

6. A MiPab comprising at least one antigen binding species selected from the group consisting of an antibody, an antibody fragment and an aptamer, wherein one antigen binding species is operably linked to a micro-bead and the ratio of antigen binding species to micro-bead is 1:1, and wherein said antigen binding species specifically binds to an antigen site of a target antigen.

7. The MiPab of claim 1, wherein the antigen binding species is an antibody.

8. The MiPab of claim 1, wherein the antigen binding species is an aptamer.

9. The MiPab of claim 1, where in the micro-particle is a micro-bead having a diameter from about 0.1 to 1.0 microns and the micro-bead comprises surface sites for attaching the antigen binding species.

10. The MiPab of claim 9, wherein the diameter of the microbead is about 0.5 microns.

11. A method of identifying a target antigen comprising the steps of
providing a plurality of MiPabs, each MiPab comprising an antigen binding species selected from the group consisting of an antibody, an antibody fragment and an aptamer, the antigen binding species having specificity for a target antigen, wherein the antigent binding species is operably linked to a micro-particle, wherein the ratio of the number of antigen binding species to each micro-bead is a predetermined, defined ratio;
applying MiPabs to a tissue preparation comprising the target antigen; and
identifying the MiPabs in which the antigen binding species are linked to the target antigen.

12. The method of claim 11, further comprising counting the MiPabs in which the antigen binding species are linked to the target antigen.

13. The method of claim 12, wherein the MiPabs are counted by light microscopy.

14. The method of claim 13, wherein the light microscopy includes image analysis algorithms.

15. The method of claim 12, further comprising converting the counted MiPabs to a measured amount of a target antigen by application of a calibration reference standard for the target antigen.

16. The method of of claim 11, wherein the antigen binding species is an antibody.

17. The MiPab of claim 11, wherein the antigen binding species is an aptamer.

18. the MiPab of claim 11, wherein the ratio is one antigen binding species to one micro-particle.

19. The MiPab of claim 11, where in the micro-particle is a micro-bead having a diameter from about 0.1 to 1.0 microns and the micro-bead comprises surface sites for attaching the antigen binding species.

20. The MiPab of claim 19, wherein the diameter of the micro-bead is about 0.5 microns.
